(19) United States
(12) Patent Application Publication
(10) Pub. No.: US 2026/0083429 A1
(43) Pub. Date: Mar. 26, 2026

(54) ARTIFICIAL INTELLIGENCE SYSTEMS FOR GENERATING ULTRASOUND IMAGING WORKFLOW STEPS

(71) Applicant: GE Precision Healthcare LLC, Waukesha, WI (US)

(72) Inventors: Hongjian Jiang, Wuxi (CN); Helene Houle, San Jose, CA (US); Hui Luo, Wuxi (CN)

(73) Assignee: GE Precision Healthcare LLC, Waukesha, WI (US)

(21) Appl. No.: 18/892,205

(22) Filed: Sep. 20, 2024

(51) Int. Cl. A61B 8/08; A61B 8/00; G06F 40/40; G06V 10/12; G06V 20/50; G16H 10/60

(52) U.S. Cl. CPC A61B 8/085; A61B 8/463; A61B 8/467; A61B 8/54; G06F 40/40; G06V 10/12; G06V 20/50; G16H 10/60; G06V 2201/03

(57) ABSTRACT

Systems are provided for assisting an ultrasound imaging workflow by predicting next steps in the workflow using artificial intelligence (AI). In one example, an ultrasound imaging system includes an ultrasound probe, an image processing circuit configured to receive image data obtained using the ultrasound probe and identify an image characteristic based on the image data, an entity recognition circuit configured to convert the identified image characteristic into an AI model input, an AI processing circuit configured to apply the AI model input into an AI model, the entity recognition circuit configured to convert an AI model output into a control signal, and a control circuit configured to control a display of the ultrasound imaging system based on the control signal, where controlling the display includes causing the display to display a plurality of recommended next steps for an operator of the ultrasound imaging system to perform in the workflow.

### FIELD

Embodiments of the subject matter disclosed herein relate to ultrasound imaging, and more particularly, to predicting next steps in an ultrasound imaging workflow using a large language model.

### BACKGROUND

During a medical imaging workflow, a plurality of medical images of a patient are obtained by a technician, such as a sonographer, to measure or detect various aspects of anatomical features present within the medical images. These images and measurements are subsequently analyzed by a clinician, such as a cardiologist or radiologist, to observe a condition or to identify any abnormalities.

### SUMMARY

An embodiment relates to an ultrasound imaging system. The ultrasound imaging system includes an ultrasound probe. The ultrasound probe includes a transducer configured to transmit and receive an ultrasound signal, a matching layer configured to have an acoustic impedance between a tissue to be imaged and a material of the transducer, and a damping block configured to absorb ultrasound energy. The ultrasound imaging system includes an image processing circuit configured to receive image data obtained using the ultrasound probe, and perform an image recognition process to identify an image characteristic based on the image data. The ultrasound imaging system includes an entity recognition circuit configured to convert the identified image characteristic into an artificial intelligence (AI) model input. The ultrasound imaging system includes an AI processing circuit configured to receive the AI model input, apply the AI model input to an AI model, and output an AI model output. The entity recognition circuit is also configured to convert the AI model output into a control signal. The ultrasound imaging system includes a control circuit configured to receive the control signal and to control a display of the ultrasound imaging system, where controlling the display comprises causing the display to display a plurality of recommended next steps for an operator of the ultrasound imaging system to perform.

Another embodiment relates to an ultrasound imaging system. The ultrasound imaging system includes an ultrasound probe and a processing circuit. The ultrasound probe includes a transducer configured to transmit and receive an ultrasound signal, a matching layer configured to have an acoustic impedance between a tissue to be imaged and a material of the transducer, and a damping block configured to absorb ultrasound energy. The processing circuit includes a processor coupled to a memory device, and the memory device stores instructions thereon that, when executed, cause the processing circuit to perform operations including receiving image data obtained using the ultrasound probe, performing an image recognition process to identify an image characteristic based on the image data, converting the identified image characteristic into an artificial intelligence (AI) model input, applying the AI model input to an AI model configured to generate an AI model output, converting the AI model output into a control signal, and controlling a display of the ultrasound imaging system based on the control signal, where controlling the display comprises causing the display to display a plurality of recommended next steps for an operator of the ultrasound imaging system to perform.

Another embodiment relates to a method. The method includes receiving, by an image processing circuit, image data obtained using an ultrasound probe. The method includes performing, by the image processing circuit, an image recognition process to identify an image characteristic based on the image data. The method includes converting, by an entity recognition circuit, the identified image characteristic into an artificial intelligence (AI) model input. The method includes receiving, by an AI processing circuit, the AI model input. The method includes applying, by the AI processing circuit, the AI model input to an AI model. The method includes outputting, by the AI processing circuit, an AI model output. The method includes converting, by the entity recognition circuit, the AI model output into a control signal. The method includes receiving, by a control circuit, the control signal. The method includes controlling, by the control circuit, a display of an ultrasound imaging system based on the control signal, where controlling the display comprises causing the display to display a plurality of recommended next steps for an operator of the ultrasound imaging system to perform.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of an imaging processing circuit used in the ultrasound imaging system of FIG. 1, according to an example embodiment.

FIG. 3 is a block diagram of an artificial intelligence circuit used in the ultrasound imaging system of FIG. 1, according to an example embodiment.

FIG. 4 is a block diagram of an entity recognition circuit used in the ultrasound imaging system of FIG. 1, according to an example embodiment.

FIG. 5 is a flow chart illustrating a method for providing intelligent recommendations during an ultrasound imaging workflow using the ultrasound imaging system of FIG. 1, according to an example embodiment.

FIG. 6 is an illustration of a user interface with options for executing an ultrasound imaging workflow, according to an example embodiment.

FIG. 7 is an illustration of a first ultrasound image from a first step in an ultrasound imaging workflow, according to an example embodiment.

FIG. 8 is an illustration of the first ultrasound image of FIG. 7 with intelligent recommendations for a second step in the ultrasound imaging workflow, according to an example embodiment.

FIG. 9 is an illustration of a second ultrasound image from the second step in the ultrasound imaging workflow based on a selected action from the intelligent recommendations of FIG. 8, and the user interface of FIG. 6 with options configured to execute the intelligent recommendations, according to an example embodiment.

FIG. 10 is an illustration of the second ultrasound image of FIG. 9 with suggested actions for a third step in the ultrasound imaging workflow, according to an example embodiment.

### DETAILED DESCRIPTION

Referring generally to the figures, an artificial intelligence (AI) system and methods for generating ultrasound imaging workflow steps are disclosed. The systems and methods disclosed herein use various processing circuits, such as an imaging processing circuit, an entity recognition circuit, an AI circuit, and a control circuit, to generate intelligent recommendations for next steps in an ultrasound imaging workflow. The ultrasound imaging system can execute a selected next step (e.g., selected by an operator of the ultrasound imaging system, such as a sonographer) from the intelligent recommendations using image processing-based automation. That is, the execution of the selected next step performs all system operations involved in the selected next step (e.g., setting acquisition parameters, mode changes, measurements, etc.), and then a next round of intelligent recommendation are generated.

Currently, a lack of standardized practices regarding how to conduct and interpret ultrasound examinations causes inconsistencies in ultrasound imaging workflows. In turn, such inconsistencies may have considerable effects on a patient's health due to errors in diagnosis and/or treatment planning. Furthermore, currently available assistive technology in the ultrasound domain adhere to fixed workflows, thus disabling flexibility between sonographers and patients, both of whom have diverse needs and preferences regarding ultrasound examinations. Current assistive technology also requires a template to be set up prior to an examination, which is time consuming and inefficient.

The ultrasound industry faces additional constraints due to busy schedules of sonographers, limited appointment times, high patient volumes, scheduling conflicts, and so on. These constraints place additional pressure on healthcare professionals, which increases the desirability of efficient ultrasound imaging workflows. For example, the additional pressure faced by sonographers may lead to rushed and/or incomplete ultrasound examinations, which can in turn, as mentioned above, be detrimental to a patient's health.

In addition to the inconsistencies and pressures associated with ultrasound imaging described above, ultrasound technicians (e.g., sonographers) may receive inadequate training and/or possess inadequate experience in performing and interpreting ultrasound examinations. Such inadequacies among sonographers may result in errors, delays, and further inconsistencies within the ultrasound imaging workflow. Therefore, a streamlined system and method for executing ultrasound imaging workflows is desirable in the ultrasound domain.

Beneficially, the implementations described herein provide assistive technology configured to provide recommendations and suggestions for ultrasound workflow steps using artificial intelligence (AI). The use of AI minimizes inconsistencies across ultrasound imaging workflows, and intelligent recommendations ensure that sonographers capture a complete ultrasound examination. Therefore, the systems and methods described herein assist sonographers in completing thorough ultrasound examinations efficiently and uniformly.

Furthermore, eliminating the inconsistencies among how ultrasound examinations are conducted and interpreted leads to less confusion among healthcare professionals (e.g., sonographers, radiologists, physicians, and other healthcare professionals), and minimizes errors in diagnosis and/or treatment planning.

The systems and methods described herein address a technical problem within ultrasound imaging systems by providing automated next steps in the ultrasound imaging workflows. That is, options for next steps in an ultrasound examination are presented to a sonographer, and the ultrasound imaging system is configured to perform operations when a next step is selected by the sonographer (e.g., by engaging with a selectable element on a touch screen, by clicking a button on a user interface, etc.). The selection prompts the ultrasound imaging system to automatically perform associated operations involved in the selected next step. Such improvements to ultrasound imaging systems minimize repetitive strain injuries experienced by sonographers by reducing the amount of input required to perform the ultrasound imaging workflow.

Further, the reduced amount of input required of sonographers results in a more efficient ultrasound examination by reducing time spent locating buttons, interface elements, measurement tools, etc., and by reducing a number of clicks required to perform next steps in the ultrasound imaging workflow. That is, the systems and methods described herein provide for multiple functions of the ultrasound imaging system to be embedded in a single selection of a recommended next step. This embedded functionality also minimizes complicated operations/interactions required of a sonographer with the ultrasound imaging system. By simplifying the ultrasound imaging workflow in this way, the learning curve associated with ultrasound examinations is flattened, allowing sonographers with less experience and/or training to perform thorough and efficient ultrasound examinations using the systems and methods described herein.

The implementations of the present disclosure provide a novel user interface and functionality within ultrasound imaging systems and workflows, where the ultrasound imaging system dynamically presents live, real-time intelligent guidance to the sonographer including multiple options for potential next steps in the workflow. Beneficially, these potential next steps may be performed without prior exam configuration. With the solution to ultrasound imaging systems described herein, the intelligent guidance is provided to the sonographer based on the initial image results during an ultrasound examination using an AI model (e.g., a large language model (LLM) using retrieval augmented generation (RAG) technology). For example, the AI model is configured to retrieve ultrasound domain information (e.g., published ultrasound guidelines from medical societies and/or a product user manual, etc.) for use in providing the intelligent guidance.

Unlike existing technology, the systems and methods disclosed herein provide a flexible ultrasound imaging workflow by generating dynamic instructions for the sonographer based on a personalized patient imaging status. That is, multiple variations of options for potential next steps are recommended to the sonographer, allowing for variations among sonographer preferences, patient preferences, examination purposes, ultrasound protocols, etc. The systems and methods described herein, however, maintain the option for a standard ultrasound imaging workflow by allowing the sonographer to dismiss the intelligent recommendations and proceed with a standard operation of the ultrasound imaging system.

The implementations described herein address a technical problem by providing enhanced data integration and analysis capabilities, which deliver a particular technical solution that streamlines and refines generation and transmittal of ultrasound imaging workflow steps. The systems described herein are implemented to improve how data is synthesized and utilized from various sources that provide information relating to an ultrasound imaging procedure. By integrating data related to a specific procedure, technician, patient, and so on, these systems provide real-time, intelligent recommendations regarding next steps to perform during an ultrasound imaging workflow. For example, the implementations can provide a recommendation to a sonographer based on anatomical features of interest for a particular ultrasound imaging procedure (e.g., an echocardiogram). In another example, the implementations can provide an actionable communication that enables the sonographer to update, for example, a setting or configuration of an ultrasound imaging system based on recommended next steps in the ultrasound imaging workflow. Accordingly, this approach provides a specific technical improvement to various technical problems, including those set forth herein.

The systems described herein may also reduce processing power by performing various processing operations simultaneously to provide intelligent recommendations regarding next steps in an ultrasound imaging workflow, rather than performing a plurality of processing operations individually and consuming unnecessary processing power. Furthermore, the systems as described herein generate next steps for a sonographer to perform in order to execute a complete ultrasound scan given various pieces of contextual information (e.g., industry standards, sonographer preferences, patient medical history, etc.). That is, the systems as described herein are trained to identify imaging parameters and operations required to obtain a complete scan in a given ultrasound procedure, therefore ensuring that the scan is complete prior to attempting to process the ultrasound images. This consideration of contextual information when generating recommended next steps in the ultrasound imaging workflow reduces processing power by avoiding collection of unnecessary ultrasound data and submission of an incomplete scan, which can cause the sonographer to have to capture additional images during a successive scan.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

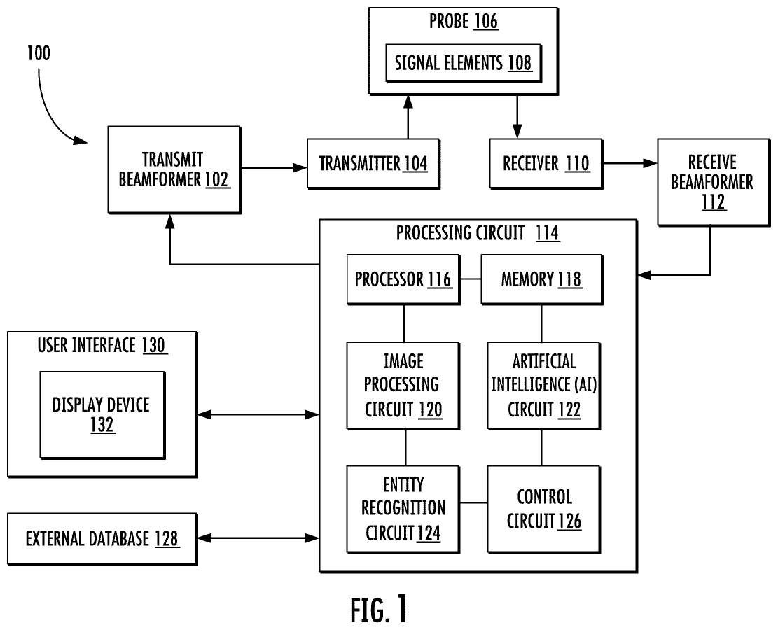

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

An example of a procedure performed using the ultrasound imaging system 100 may be an echocardiogram. Echocardiograms are performed to detect heart abnormalities in a patient by collecting and processing ultrasound data (e.g., using the ultrasound imaging system 100, as described herein). During an echocardiogram, a sonographer follows a particular imaging protocol specific to echocardiography. The echocardiography-specific imaging protocol ensures that the heart is thoroughly captured by the ultrasound data and that the processing of the ultrasound data is focused on detecting heart abnormalities. The sonographer collects the ultrasound data by navigating a probe (e.g., probe 106, as described below) over the patient's chest until a sufficient volume of ultrasound images are collected. The collected images are stored in a central storage device (e.g., memory 118) and analyzed by the sonographer. The sonographer generates a set of measurements from the images (e.g., 50-100 records), and the images and measurements are collectively reviewed by a cardiologist. The cardiologist provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data and 2D color flow data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during an echocardiogram, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, an artificial intelligence (AI) circuit 122, an entity recognition circuit 124, and a control circuit 126. In this way, the processing circuit 114 may be structured or configured to execute or implement the instructions, commands, and/or control processes described herein with respect to the processor 116, the memory 118, the image processing circuit 120, the AI circuit 122, the entity recognition circuit 124, and the control circuit 126.

The processor 116 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, as described herein, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

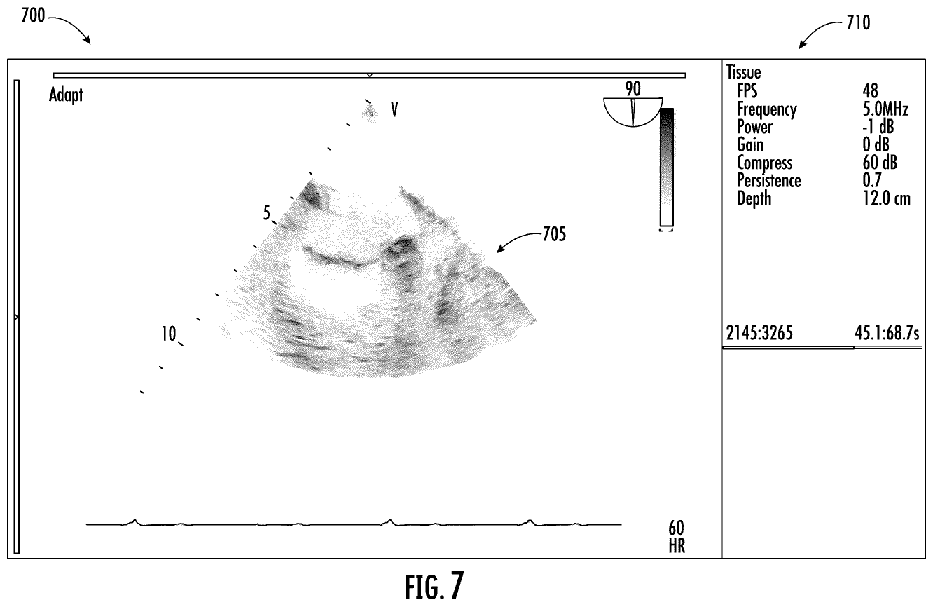

The processor 116 may also be in electronic communication with the display device 132 such that the processor 116 may process ultrasound data obtained by the probe 106 and generate images to display on the display device 132 (e.g., first ultrasound image 705 and/or second ultrasound image 905, as described below with reference to FIGS. 7-10).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

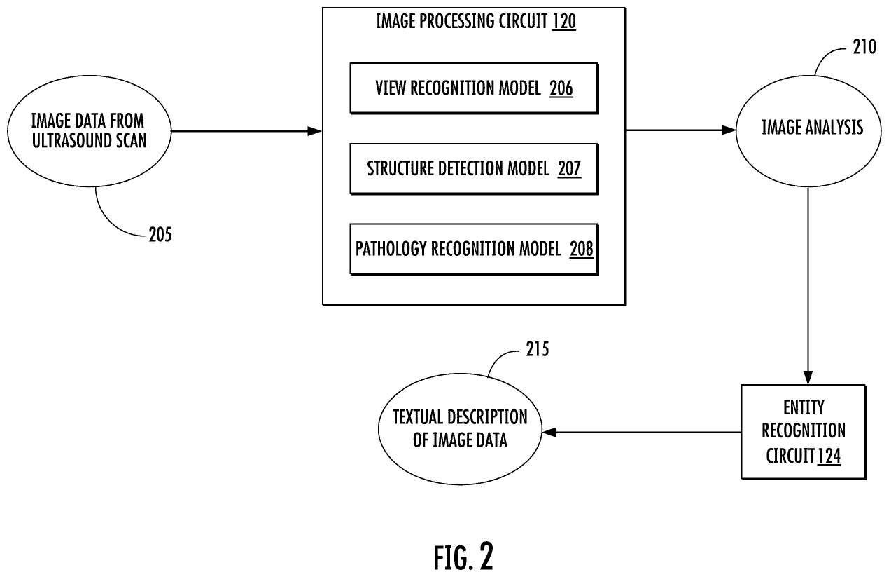

The processing circuit 114 also includes the image processing circuit 120, the AI circuit 122, the entity recognition circuit 124, and the control circuit 126. Each of the image processing circuit 120, the AI circuit 122, the entity recognition circuit 124, and the control circuit 126 are configured to facilitate predicting and performing ultrasound imaging workflow steps, and are each described in greater detail below with reference to FIGS. 2-5.

The ultrasound imaging system 100 may also include an external database 128 and a user interface 130. The external database 128 refers to a database from which the processing circuit 114 (e.g., the AI circuit 122) retrieves information used in providing intelligent guidance during an ultrasound imaging workflow. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the AI circuit 122 may retrieve clinical guidelines, standard practices, medical literature, medical textbooks, published research, and previous case studies related to the implementation and/or procedure. For example, if the ultrasound imaging system 100 is being used in a hospital setting to perform an LAA closure procedure, the AI circuit 122 may retrieve clinical guidelines and standard practices related to the hospital setting and the LAA closure procedure. Continuing with this example, the AI circuit 122 may also retrieve information from the medical literature, medical textbooks, published research, and previous case studies related to cardiac anatomy and the LAA closure procedure.

The user interface 130 may be used by a sonographer or other clinician to control operation of the ultrasound imaging system 100. For example, the sonographer may use the user interface 130 to control the input of patient data, to change a scanning or display parameter, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

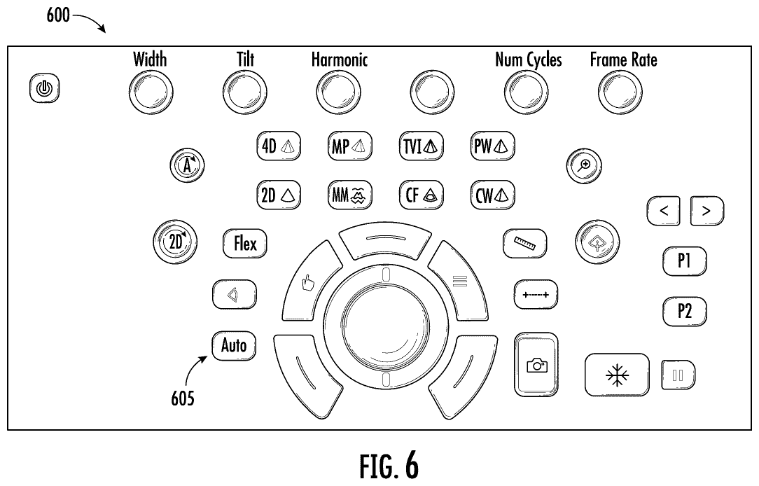

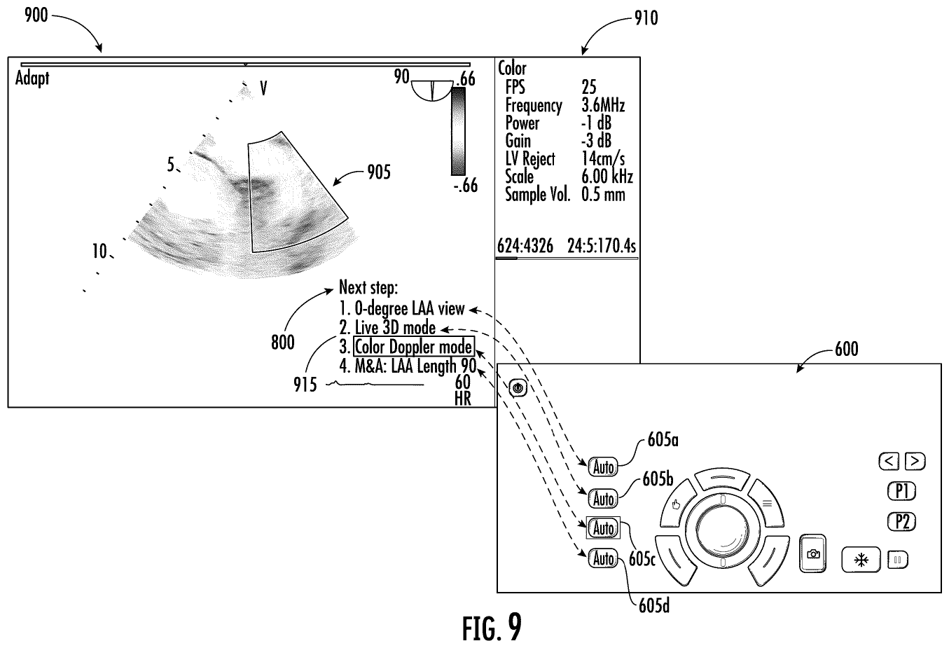

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons. As described below, FIGS. 6 and 9 illustrate a GUI 600 where at least some of the physical controls of the user interface 130 are presented by various user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermine distance (e.g., five feet, six inches, ten centimeters, etc.) of the proximity sensor. In various embodiments, the proximity sensor may be located on the display device 132 or as part of a touch screen that is separate from the display device 132.

Referring to FIG. 2, the image processing circuit 120 of the ultrasound imaging system 100 is shown in greater detail. As shown, the image processing circuit 120 receives image data 205 from an ultrasound scan. The image data 205 refers to ultrasound data collected by the probe 106 while performing an ultrasound examination on a patient. For example, the image data 205 may be collected during an echocardiogram procedure and may therefore include various images of a patient's heart.

The image data 205 is received and processed by the image processing circuit 120 during an image recognition process. In some embodiments, the image data 205 may be retrieved by the image processing circuit 120 from the memory 118. The image data 205 may include a first set of images from a scanning session (e.g., the echocardiogram procedure). The first set of images may be taken with the ultrasound imaging system 100 in a first mode of operation (e.g., B-mode, color flow Doppler mode, M-mode, color M-mode, spectral Doppler, elastography, TVI, strain, strain rate, etc.). In some embodiments, the first set of images may relate to a specific anatomical feature and/or region of the patient.

The image processing circuit 120 may include multiple deep learning-based models configured to analyze the image data 205. In some embodiments, the image processing circuit may include a view recognition model 206, a structure detection model 207, and a pathology recognition model 208. The view recognition model 206 may be configured to identify a view from which the image data 205 is captured. That is, the view recognition model 206 receives the image data 205 and then identifies a view or a scan plane being imaged in the image data 205. For example, the structure detection model 207, as described below, may determine that the image data 205 depicts a heart, and the view recognition model 206 may further identify that the image data 205 depicts a two-chamber view of the heart (e.g., as opposed to a three-chamber view, a four-chamber view, etc.). In this example, the view recognition model 206 may determine that the image data 205 is taken from the ME_2CH TEE view, which stands for a mid-esophageal (ME) 2-chamber (2CH) transesophageal echocardiography (TEE) view.

The structure detection model 207 may be configured to identify an anatomical structure, feature, region, etc. captured by the image data 205. The structure detection model 207 may be configured to identify the anatomical structure using one or more algorithms (e.g., image processing algorithms such as edge detection, machine learning models, deep neural networks, etc.). In some embodiments, the structure detection model 207 may identify anatomical features such as bones, blood vessels, organs, etc., based on a shape, relative proximity, apparent depth, orientation, etc. of said features in the image data 205. Then, based on the identified anatomical features, the structure detection model 207 may be configured to determine the anatomical feature being imaged in the image data 205. For example, during an echocardiogram in which the ultrasound imaging system 100 is being used to capture images of a patient's heart, the structure detection model 207 may determine that the left atrium is being imaged based on a movement and/or orientation of the left atrium relative to surrounding structures, such as the aorta, via a deep learning classification model trained to recognize the movement and/or orientation of the left atrium, and other anatomical structures. In some embodiments, the structure detection model 207 may detect movement of structures in the image data 205 by comparing the location, shape, size, etc., of identified structures across the first set of images.

The pathology recognition model 208 may be configured to determine the presence of a pathology (e.g., an injury, disease, abnormality, etc.) in the image data 205. For example, the pathology recognition model 208 may tag the image data 205 as “pathology detected” if a pathology is present, or “no pathology” if a pathology is not present. In some embodiments, the pathology recognition model 208 may use a deep learning classification model trained to recognize various pathologies in the anatomical structure represented by the image data 205 to specify the pathology that is present. Continuing with the example of an echocardiogram, the pathology recognition model 208 may use a deep learning classification model trained to recognize cardiovascular pathologies to determine whether a pathology is present in the image data 205 from the echocardiogram. For instance, the pathology recognition model 208 may identify a blood clot in the LAA as a pathology from the image data 205.

Therefore, based on the received image data 205, the image processing circuit 120 is configured to perform an image analysis 210. For instance, in some embodiments, the image analysis 210 may include identifying an image characteristic. The image characteristic may include at least one of a mode, a view (e.g., determined by the view recognition model 206), an identification of an imaged structure (e.g., identified by the structure detection model 207), an identification of a pathology (e.g., identified by the pathology recognition model 208), an acquisition parameter, or a measurement setting associated with the image data 205. The mode may refer to the mode of operation of the ultrasound imaging system 100 (e.g., B-mode, color flow Doppler mode, M-mode, color M-mode, spectral Doppler, elastography, TVI, strain, strain rate, etc.). In some embodiments, the mode, the acquisition parameter, and/or the measurement setting may be received from the memory 118.

In some embodiments, the image processing circuit 120 may be configured to generate a textual description of the image data 215. In such embodiments, the textual description of the image data 215 may be included in the image analysis 210. That is, the image analysis 210 may include the identified image characteristic and the textual description of the image data 215. In some embodiments, and as shown in FIG. 2, the entity recognition circuit 124 may generate the textual description of the image data 215. In such embodiments, the image analysis 210 may include the identified image characteristic, which may be used an input into the entity recognition circuit 124. The entity recognition circuit 124 then receives the identified image characteristic as the input and determines the textual description of image data 215 based on the input. For example, where the identified image characteristics include the mode, the view, the imaged structure, and the acquisition parameter, the textual description of the image data 215 may be “Step 1: A B-mode image with a ME_2CH view, where a LAA is visible, and where the transducer angle is 90-degrees.”

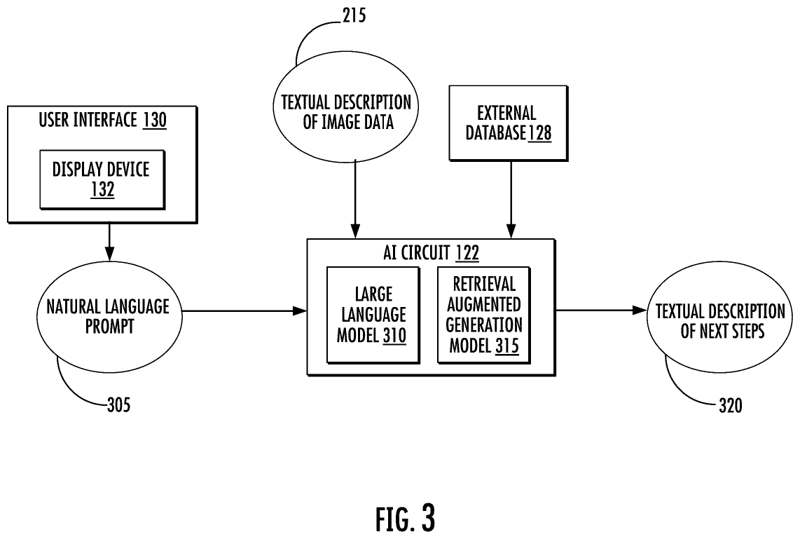

Referring to FIG. 3, the AI circuit 122 of the ultrasound imaging system 100 is shown in greater detail. The AI circuit 122 is shown to receive the textual description of the image data 215 as an AI model input (e.g., generated by the image processing circuit 120 and/or the entity recognition circuit 124, as described above with reference to FIG. 2). That is, the AI model input includes the textual description of image characteristics identified by the image processing circuit 120 from the image data 205. For example, the AI model input may include “Step 1: A B-mode image with a ME_2CH view, where a LAA is visible, and where the transducer angle is 90-degrees.”

In some embodiments, the AI circuit 122 may also receive a natural language prompt 305 from the user interface 130, as shown in FIG. 3. The natural language prompt 305 may include a request for intelligent guidance regarding recommended next steps in the ultrasound imaging workflow based on a current status of the ultrasound imaging workflow. For example, the natural language prompt 305 may be “The current step is a B-mode image with a ME_2CH view, where a LAA is visible. The transducer angle is 90. What are the next steps?” Alternatively, the natural language prompt 305 may omit the description of the current status of the ultrasound imaging workflow (e.g., “The current step is a B-mode image with a ME_2CH view, where a LAA is visible. The transducer angle is 90.”), as such information is otherwise provided as an input to the AI circuit 122 from the textual description of the image data 215. In other words, the natural language prompt 305 may include the request for intelligent guidance alone (e.g., “What are the next steps?”, “Provide guidance regarding the next steps,” etc.). In some embodiments, the natural language prompt 305 may be received as an input from an operator of the ultrasound imaging system 100 (e.g., a sonographer) via the display device 132. For example, the input may be submitted via the touch screen or typed using the keyboard.

The AI circuit 122 may be configured to apply the textual description of the image data 215 and the natural language prompt 305 to an AI model. That is, the AI model input refers to a natural language input. Therefore, as shown in FIG. 3, the AI model may be a large language model (LLM) 310 configured to receive the natural language input. In some embodiments, the LLM 310 may be powered by a retrieval augmented generation (RAG) model 315. The RAG model 315 may be configured to access the external database 128 such that the information contained therein (e.g., clinical guidelines, standard practices, medical literature, medical textbooks, published research, or previous case studies) may be retrieved as the information relates to the AI model input. For example, if the textual description of the image data 215 states “Step 1: A B-mode image with a ME_2CH view, where a LAA is visible, and where the transducer angle is 90-degrees”, the RAG model 315 may be configured to retrieve information from the external database 128 relating to heart anatomy, LAA closure procedures, echocardiograms, etc. Therefore, with the RAG model 315, the LLM 310 may be configured to generate an output using information relating to a specific procedure (e.g., an echocardiogram), a specific setting (e.g., a hospital), a specific anatomy (e.g., a heart), and so on, without requiring a retraining of the LLM 310 for uses relating to each of the specific procedure, the specific setting, the specific anatomy, and so on.

The information relating to the AI input retrieved from the external database 128 may then be combined with the AI input (e.g., the textual description of the image data 215) to create an augmented AI model input. That is, the AI model input is augmented with the relevant information retrieved from the external database 128. The augmented AI model input may be applied as an input to the LLM 310 such that an AI model output from the LLM 310 is based on the augmented AI model input. For instance, where the AI model input is “Step 1: A B-mode image with a ME_2CH view, where a LAA is visible, and where the transducer angle is 90-degrees” using the textual description of the image data 215 alone, the augmented AI input may be “Step 1: A B-mode image with a ME_2CH view, where a LAA is visible, and where the transducer angle is 90-degrees. Preparation for an LAA closure procedure requires three-and four-chamber views, color-Doppler mode imaging, and images of the aorta.”

From the LLM 310, the AI circuit 122 may be configured to output an AI model output. In some embodiments, the AI model output includes a set of natural language instructions generated by the LLM 310. For instance, as shown in FIG. 3, the AI model output may include a textual description of next steps 320. The textual description of next steps 320 refers to a plurality of recommended next steps for the sonographer to perform in the ultrasound imaging workflow based on a current status of the workflow. For example, the plurality of recommended next steps may include a recommendation to rotate or move the ultrasound probe a certain way, a recommendation to activate a particular mode, or a recommendation to take a particular measurement. Where the input to the LLM 310 is the augmented AI model input, the output from the LLM 310 may be based on the augmented AI model input, rather than on the description of the image data 215 alone. For instance, the AI model output based on the augmented AI model input may include next steps relating to obtaining a three-chamber view, obtaining a four-chamber view, switching to the color Doppler mode, and/or imaging the aorta. Alternatively, the AI model output based on the description of the image data 215 alone may include next steps relating to switching to the M-mode, changing the transducer angle, and/or imaging the right atrium.

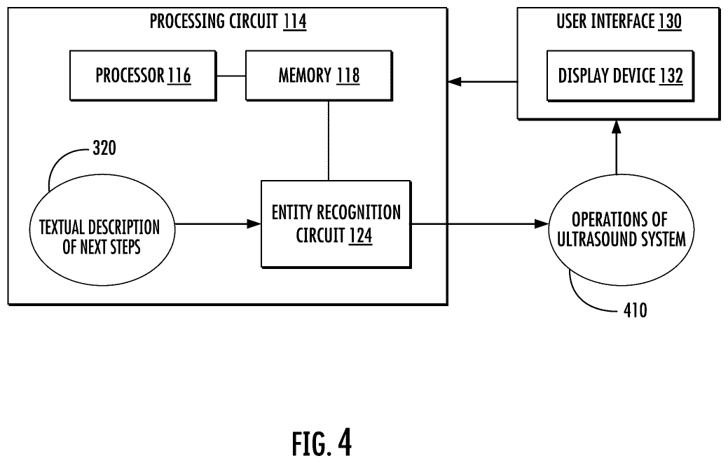

Referring to FIG. 4, the processing circuit 114 including the processor 116, the memory 118, and the entity recognition circuit 124 is shown. The entity recognition circuit 124 is shown as receiving the textual description of next steps 320 (e.g., the output from the AI circuit 122, as shown in FIG. 3). The entity recognition circuit 124 may be configured to convert the textual description of next steps 320 into operations 410 of the ultrasound imaging system 100. The operations 410 of the ultrasound imaging system 100 refer to operations/functions performable by various components of the ultrasound imaging system 100 (e.g., changing a mode of operation, updating acquisition parameters, etc.) that correspond to operations/functions included in the textual description of next steps 320. For example, if the textual description of next steps 320 includes “rotating the transducer angle to obtain the 0-degree LAA view and optimize gain settings to better visualize the LAA ostium and morphology, acquiring a full-volume 3D dataset centered on the LAA by activating a live 3D/real-time 3D model, switching to a color Doppler mode in the ME_2CH view to evaluate for any flow across the LAA ostium or into the LAA cavity, and measuring the LAA ostium diameter to evaluate the size of the LAA ostium for selection of an occlude device,” the entity recognition circuit 124 may identify the operations 410 of the ultrasound imaging system 100 to be switching to a 0-degree LAA view, switching to a live 3D mode, switching to a color Doppler mode, and measuring a length of the LAA from the 90-degree view.

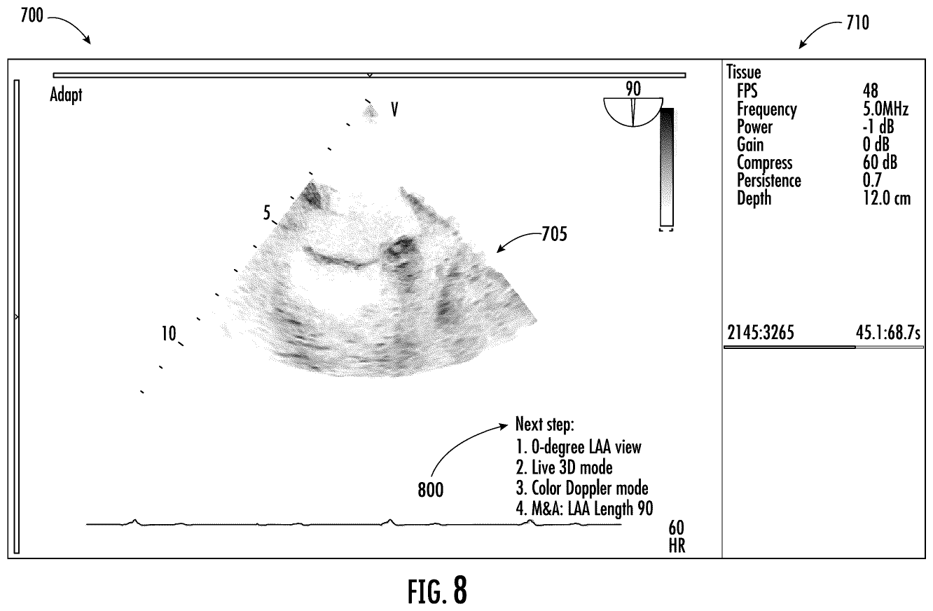

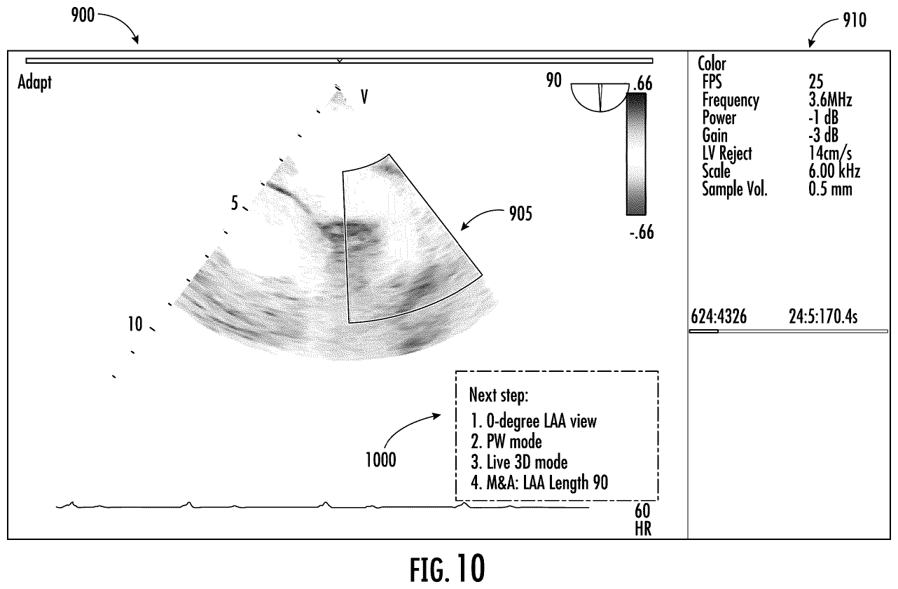

In some embodiments, each of the operations 410 may be represented by a control signal generated by the entity recognition circuit 124. The control circuit 126 may receive the control signals from the entity recognition circuit 124 and, in response, control a display of the user interface 130 (e.g., the display device 132). That is, the control circuit 126 may control the display of the user interface 130 such that the operations 410 of the ultrasound imaging system 100 may be presented to an operator of the ultrasound imaging system 100 (e.g., the sonographer) via the user interface 130. For instance, the operations 410 may be displayed as a list of options on a GUI (e.g., the options for next steps 800 as shown in FIGS. 8-9, the new options for next steps 1000 as shown in FIG. 10, etc.). In some embodiments, each of the operations 410 presented on the GUI may correspond to a selectable element (e.g., selectable elements 605a-605d on GUI 600, as shown in FIG. 9) configured to automate execution of the operations/functions of the ultrasound imaging system 100 described by the corresponding operation 410. In this way, the operator may select one of the operations 410 by selecting the corresponding selectable element on the display device 132.

As shown in FIG. 4, the processing circuit 114 may receive the selection of one of the operations 410 from the user interface 130. For example, the processing circuit 114 may receive the selection as a touch screen input on the display device 132 from the operator of the ultrasound imaging system 100 (e.g., by clicking on/tapping one of the selectable elements 605a-605d on the GUI 600). In response to receiving the selection of one of the operations 410, the processing circuit 114 prompts respective components of the ultrasound imaging system 100 to perform the operations/functions described by the selected operation. In some embodiments, the control circuit 126 may be configured to receive the selection of one of the operations 410 and, in response, change an operating characteristic (e.g., the mode, the view, the acquisition parameter, the measurement setting, etc.) of the ultrasound imaging system 100.

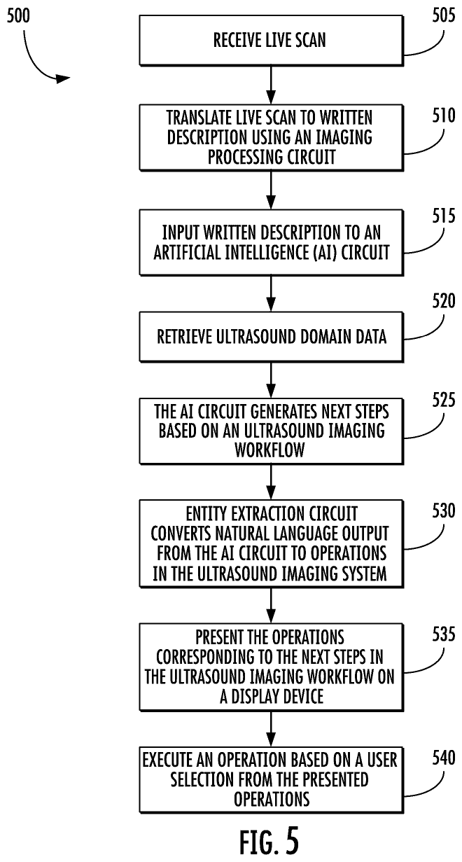

Referring to FIG. 5, a flow chart is shown illustrating a method 500 for providing intelligent recommendations during an ultrasound imaging workflow using an ultrasound imaging system. In at least one embodiment, the ultrasound imaging system referred to by method 500 is the ultrasound imaging system 100 described above with reference to FIGS. 1-4, and method 500 may be implemented by the ultrasound imaging system 100. In some embodiments, method 500 may be implemented as executable instructions in a memory of the ultrasound imaging system 100, such as the memory 118 of FIG. 1.

Prior to initiating a collection of ultrasound data, method 500 may begin when an operator (e.g., a sonographer, technician, or other clinician) is authenticated as an authorized user of the ultrasound imaging system 100. In some embodiments, the operator may authenticate themselves as an authorized user of the ultrasound imaging system 100 by logging in to a portal (e.g., an online application accessible via the user interface 130) associated with the environment in which the ultrasound imaging system 100 is being implemented (e.g., a hospital or other healthcare provider). For instance, the operator may log in using a unique identifier (e.g., a username, a password, a biometric scan, a pin code, etc.).

After the operator is authenticated, the operator of the ultrasound imaging system 100 may enter patient and/or procedure-specific information into the ultrasound imaging system 100 prior to the collection of ultrasound data. For example, the operator may submit patient information (e.g., identifying information such as name, date of birth, social security number, and so on, and/or medical information such as a medical history, family medical history, a current diagnosis, and so on) via the user interface 130. In some embodiments, the operator may select the patient from a list of patients (e.g., patients associated with a scheduled procedure to be performed by the operator), and the patient information may be imported to the ultrasound imaging system 100 (e.g., from a database associated with the environment in which the ultrasound imaging system 100 is being implemented, such as a hospital).

In addition to the patient information, the operator may enter (e.g., as a text entry) or otherwise select (e.g., from a drop-down list of procedures) the procedure that the operator is preparing to perform. For example, the operator may enter or select “echocardiogram” as the procedure. Additionally, the operator may enter or otherwise select any known pathologies or other medical conditions that may be relevant to the procedure. For instance, the operator may be performing an ultrasound examination on the patient in preparation for an LAA closure procedure, and such information may be entered into the ultrasound imaging system 100 prior to the collection of ultrasound data. In this way, the ultrasound imaging system 100 may be configured to prepare intelligent guidance regarding the ultrasound imaging workflow, as described herein, specific to data that may be relevant to the LAA closure procedure (e.g., sufficient imaging of the patient's heart and, more specifically, the left atrium).

Additionally, prior to initiating the collection of ultrasound data, method 500 may include the operator of the ultrasound imaging system 100 selecting an operating mode of the ultrasound imaging system 100. In some embodiments, the operating mode may refer to an imaging mode of an ultrasound probe (e.g., probe 106). For example, the operating mode may include any of the imaging modes described above, such as the B-mode, color flow Doppler mode, M-mode, Color M-mode, spectral Doppler, Elastography, TVI, strain, strain rate, and the like. In some embodiments, the operator may select the imaging mode of the probe 106 via a user device (e.g., the user interface 130). For example, the operator may select the imaging mode via a GUI presented on a touch screen display device (e.g., display device 132). Alternatively or additionally, the operator may engage with a button or other physical control located on the probe 106 and configured to control the operating mode. Each operating mode may correspond to a particular method of operation of the probe 106. For example, the operating mode may be configured to control signals transmitted to the signal elements 108 of the probe 106 and/or process signals received from the signal elements 108 of the probe 106 according to a particular method.

As shown in FIG. 5, at step 505, method 500 may include receiving ultrasound data from a live scan performed by the ultrasound imaging system 100 using the probe 106. The ultrasound data may refer to a set of ultrasound images depicting a patient's anatomy (e.g., including first ultrasound image 705, as described below). After the ultrasound data is collected by the probe 106 and converted to image data by the processing circuit 114 (e.g., by the processor 116, as described above), the images may be stored in the memory 118. The image data may include individual images and/or a cine loop (e.g., a series of five images, ten images, twenty images, etc.). The cine loop refers to a series of images relating to an anatomical region captured sequentially by the probe 106. In some embodiments, additional image data may be stored continuously in the memory 118 as a scanning session progresses and new ultrasound data is obtained. Where the memory 118 has a limited capacity (e.g., storage for 100 images), in some embodiments, older images in the set of ultrasound images may be replaced by new images as new ultrasound data is obtained by the probe 106 and stored in the memory 118.

At step 510, the image data from the live scan received at step 505 is translated into a written description. The written description may be generated by the image processing circuit 120 and/or the entity recognition circuit 124, as described above with reference to FIG. 2. That is, the written description generated at step 510 may be the textual description of image data 215, as shown in FIG. 2. For example, the written description may include image characteristics such as an imaging mode (e.g., B mode), a view (e.g., ME_2CH view), an anatomical structure (e.g., LAA), a pathology (e.g., myocarditis), and/or an acquisition parameter (e.g., 90-degree transducer angle). As described with reference to FIG. 2, the view may be determined by the view recognition model 206, the anatomical structure may be determined by the structure detection model 207, and the pathology may be determined by the pathology recognition model 208. The image mode and the acquisition parameter may be retrieved from the memory 118. As an example, the written description of the image data from the live scan may be “Step 1: A B-mode image with a ME_2CH view, where a LAA is visible. The transducer angle is 90-degrees.” In this example, the written description includes a current status (e.g., step) of the ultrasound imaging workflow. That is, “Step 1” may indicate that the operator has captured a first set of images of a first anatomical region of the patient in a first imaging mode, and that steps 2-N are to follow in the ultrasound imaging workflow (e.g., where “N” is the last step in the ultrasound imaging workflow prior to completing a sufficient ultrasound examination).

The written description generated at step 510 may then be used as an input into an AI circuit at step 515. The AI circuit may be the AI circuit 122 and may include the LLM 310 and the RAG model 315, as shown in FIG. 3. In some embodiments, the written description (e.g., the textual description of image data 215) may be combined with a natural language prompt (e.g., the natural language prompt 305) and fed into the LLM 310. For example, the input received by the LLM 310 at step 515 may be of the generic form, “For a specific diagnosis/procedure, please help to derive the next step to perform in an ultrasound scan based on the current step. You should provide N ‘configurable’ options.” As a specific example, the prompt may be “For an LAA closure procedure, please help to derive the next step to perform in an ultrasound scan based on the current step. You should provide 3 options.” In this example, the current step may be identified based on information contained within the written description generated at step 510 (e.g., “Step 1: A B-mode image with a ME_2CH view, where a LAA is visible. The transducer angle is 90-degrees.”).

At step 520, ultrasound domain data is retrieved. The ultrasound domain data refers to contextual information relating to the ultrasound imaging workflow being performed. For example, the ultrasound domain data may include the information stored in the external database 128, as described above with reference to FIG. 1. That is, the ultrasound domain data may include clinical guidelines, standard practices, medical literature, medical textbooks, published research, previous case studies, and so on, relating to the ultrasound imaging workflow. Relevant information may relate to a specific procedure (e.g., an LAA closure), a specific setting in which the ultrasound imaging workflow is being formed (e.g., a hospital), an anatomical feature being imaged (e.g., a heart), and so on. As described in greater detail with reference to FIG. 3, the ultrasound domain data may be retrieved at step 520 by the RAG model 315.

In some embodiments, documents included in the external database 128 may have documents embeddings configured to describe the information contained therein. For example, a document titled “Imaging Modalities Used for Assessing Left Atrial Appendage Closure” may have various document embeddings relating to ultrasound imaging modalities, heart anatomy (more specifically, the LAA), LAA closure procedures, and so on. The document embeddings may be recognizable by the RAG model 315 such that the RAG model 315 is configured to retrieve relevant documents from the external database 128 based on the document embeddings and the written description. For instance, where the written description is “Step 1: A B-mode image with a ME_2CH view, where a LAA is visible. The transducer angle is 90-degrees.”, the RAG model 315 may retrieve the “Imaging Modalities Used for Assessing Left Atrial Appendage Closure” document from the external database 128 based on the document embeddings that relate to the information included in the written description (e.g., the ultrasound imaging modalities and the LAA anatomy).

After the written description is received by the AI circuit at step 515 and the ultrasound domain data is retrieved at step 520, method 500 may proceed when the AI circuit generates next steps based on the ultrasound imaging workflow at step 525. The next steps generated at step 525 may refer to the textual description of next steps 320. As described with reference to FIG. 3, the next steps may be generated by the LLM 310 based on the written description (e.g., the textual description of the image data 215) and the ultrasound domain data (e.g., the information retrieved by the RAG model 315 from the external database 128). A generic output generated in response to the input received by the LLM 310 may be of the form, “Here are N steps to perform after the current step: . . . ”. In some embodiments, the output generated by the AI circuit at step 525 may also include a summary of the current status of the ultrasound imaging workflow (e.g., the last step performed in the workflow).

For instance, the output may recite “Given that the current step is a B-mode image in the mid-esophageal 2-chamber view (ME_2CH) at 90 degrees, where the LAA is visible and healthy, here are four options for the next steps on the ultrasound scanner: . . . ” and may proceed by outputting various options of next steps to perform in the ultrasound imaging workflow. In this instance, the four options for the next steps may include (1) rotating the transducer angle to obtain the 0-degree LAA view and optimize gain settings to better visualize the LAA ostium and morphology, (2) acquiring a full-volume 3D dataset centered on the LAA by activating a live 3D/real-time 3D model, (3) switching to a color Doppler mode in the ME_2CH view to evaluate for any flow across the LAA ostium or into the LAA cavity, and (4) measuring the LAA ostium diameter to evaluate the size of the LAA ostium for selection of an occlude device.

At step 530, an entity extraction circuit converts the natural language output from the AI circuit 122 to corresponding operations in the ultrasound imaging system 100. For instance, the entity extraction circuit may be the entity recognition circuit 124, as described above with reference to FIG. 4. In this instance, the entity recognition circuit 124 converts the natural language output from the LLM 310 (e.g., the textual description of next steps 320) into operations of the ultrasound imaging system 100. The operations generated at step 530 may refer to the operations 410, also described above with reference to FIG. 4. As described above with reference to FIG. 4, the entity recognition circuit 124 may be configured to generate control signals corresponding to each of the options for next steps such that the operations associated with each option (e.g., a mode change, an update to an acquisition parameter, activating a measurement tool, etc.) may be embedded into the control signal.

Continuing with the example introduced above, where the natural language output from the LLM 310 begins with “Given that the current step is a B-mode image in the mid-esophageal 2-chamber view (ME_2CH) at 90 degrees, where the LAA is visible and healthy, here are four options for the next steps on the ultrasound scanner: . . . ”, the entity recognition circuit 124 may be configured to convert each of the four options for the next steps into corresponding operations performable by the ultrasound imaging system 100. That is, the entity recognition circuit 124 may be trained to identify a button and/or interface element in the ultrasound imaging system 100 configured to change the acquisition parameters in order to rotate the transducer angle to obtain the 0-degree LAA view, as described by the first recommended option for next steps. As another example, the entity recognition circuit 124 may be trained to identify a button and/or interface element in the ultrasound imaging system 100 configured to change a mode of operation in order to activate a live 3D/real-time 3D model. Further, the entity recognition circuit 124 may be trained to identify a button and/or interface element in the ultrasound imaging system 100 configured to change a mode of operation in order to switch to the color Doppler mode in the ME_2CH view. As yet another example, and based on the fourth recommended option for next steps mentioned above, the entity recognition circuit 124 may be trained to identify a button and/or interface element in the ultrasound imaging system 100 configured to activate a measurement tool used to measure the LAA ostium diameter. Each of these operations may be embedded into a control signal corresponding to each of the options for next steps.

At step 535, the operations corresponding to the next steps in the ultrasound imaging workflow (e.g., determined by the entity extraction circuit at step 530) may be presented on a display device. In some embodiments, the operations may be presented via the display device 132 of the user interface 130. For example, the operations may be presented as the options for next steps 800, as shown in FIGS. 8 and 9. In some embodiments, the control circuit 126 may be configured to display the operations corresponding to each of the recommended next steps based on receiving the control signal from the entity recognition circuit 124. Further, a configuration of the display of the operations on the display device 132 may be customized for the operator of the ultrasound imaging system 100 performing the ultrasound examination based on operator settings/preferences. In some implementations, an operator preference may relate to an amount of detail relating to each of the operations included in the display. For example, where a recommended next step generated by the LLM 310 includes switching to a color Doppler mode in the ME_2CH view to evaluate for any flow across the LAA ostium or into the LAA cavity, the display on the ultrasound imaging system 100 may simply include “switch to color Doppler mode,” thereby omitting the additional explanation for the recommended next steps generated by the LLM 310. Alternatively, an operator of the ultrasound imaging system 100 may prefer to receive a detailed explanation/reason for each of the recommended next steps with the display of the operations at step 535.

According to some embodiments, the control circuit 126 may be configured to display the operations in real-time with respect to the processing of the image data (e.g., by the image processing circuit 120) received during the ultrasound scan. That is, as additional data is collected using the probe 106 and processed by the image processing circuit 120, the operations displayed via the ultrasound imaging system 100 may be updated in real-time. As an example, if one of the operations displayed on the user interface 130 includes collecting images of a patient's aorta, and the image processing circuit 120 (e.g., using the structure detection model 207) detects that a series of images of the patient's aorta have been collected thereafter, the control circuit 126 may receive an instruction to remove the operation suggesting collecting the images of the patient's aorta.

Step 540 of the method 500 involves executing an operation based on a user selection from the operations presented at step 535. In some embodiments, the user selection may be a user input received via the user interface 130. For example, the user input may refer to a selection (e.g., a tap) of an interface element on the display device 132 representing one of the presented operations (e.g., one of selectable elements 605a-605d, as described below with reference to FIG. 9). In some embodiments, after receiving the user selection from the presented options, the control circuit 126 may be configured to execute the operation at step 540. That is, the control circuit 126 may be configured to execute the operation by changing an operating characteristic (e.g., a mode, a view, an acquisition parameter, a measurement setting, etc.) of the ultrasound imaging system 100. For example, if the user selects an option to switch to color Doppler mode, the control circuit 126 may receive a control signal (e.g., the control signal generated by the entity recognition circuit 124, as described above) associated with the selected option, and the control signal may cause the control circuit 126 to change the operating mode of the ultrasound imaging system 100 to the color Doppler mode.

Following the execution of the operation at step 540, the method 500 may repeat as an iterative process based on the executed operation. That is, the AI circuit 122 may be configured to generate recommended next steps (e.g., step 525) based on the updated status of the ultrasound imaging workflow after the completion of step 540. For example, as described above, the written description generated at step 510 in a first iteration of method 500 may be “Step 1: A B-mode image with a ME_2CH view, where a LAA is visible. The transducer angle is 90-degrees.” Thus, the performance of the first iteration of method 500 (e.g., as described herein) may follow from such a written description. However, after executing the operation at step 540 during the first iteration, such as switching the operating mode of the ultrasound imaging system 100 to a color Doppler mode, method 500 may repeat based on an updated written description generated at step 510 during a second iteration, such as “Step 2: A color Doppler mode image with a ME_2CH view, where a LAA is visible. The transducer angle is 90-degrees.” In this instance, performance of the second iteration of method 500 may follow based on the updated written description.

In the second iteration of method 500, the recommended next step chosen during the first iteration may be replaced with a new recommendation based on the updated status of the ultrasound imaging workflow. For example, from the options generated at step 525 during the first iteration, as described above, the AI circuit 122 may replace the option to switch to color Doppler mode (which was executed at step 540) to an option to switch to a pulsed-wave (PW) Doppler mode in the second iteration. The remainder of the recommended next steps from step 525 of the first iteration (e.g., rotating the transducer angle, activating a live 3D/real-time 3D model, and measuring the LAA ostium diameter) may remain in the recommended next steps of each iteration of method 500 until each option is selected by the operator of the ultrasound imaging system 100. However, it will be appreciated that the reminder of the recommended next steps from step 525 of the first iteration may also be replaced by other recommended next steps, and provided as recommended next steps after subsequent iterations of method 500.

Referring to FIG. 6, a GUI 600 with selectable elements configured to enable a user (e.g., an operator of the ultrasound imaging system 100) to control operation of the ultrasound imaging system 100 by selection thereof is shown. In some embodiments the GUI 600 may be a GUI generated for display on the display device 132. Further, the GUI 600 may be configured as a touch screen display, such that the user may select one of the selectable elements by touching the respective location of the selectable element on the touch screen display. Alternatively or additionally, each of the selectable elements shown on the GUI 600 may be configured as hardware elements (e.g., physical buttons) included as part of the user interface 130.

As shown, the GUI 600 includes selectable element 605, which may be represented as an “Auto” button. The selectable element 605 may be configured to perform various automated operations of the ultrasound imaging system 100, depending on the ultrasound imaging workflow being executed. That is, selection of the selectable element 605 may prompt the processing circuit 114 (e.g., the control circuit 126) to instruct the ultrasound imaging system 100 to perform one or more functions/operations associated with the selectable element 605. For example, during an echocardiogram workflow, the selectable element 605 may be configured to automate switching between imaging modes (e.g., M-mode, B-mode, color Doppler mode, etc.) required during the workflow to obtain sufficient imaging of the heart. In some embodiments, the GUI 600 may include a plurality of selectable elements 605 (e.g., 605a-605d, as described below with reference to FIG. 9), each configured to automatically perform a next step in the ultrasound imaging workflow.

Referring to FIGS. 7-8, a GUI 700 displaying a first ultrasound image 705 of a first anatomical structure taken during an ultrasound examination (e.g., an echocardiogram) is shown. In some embodiments, the GUI 700 may be displayed via the display device 132 of the ultrasound imaging system 100. The first ultrasound image 705 may be obtained by the ultrasound imaging system 100 shown by FIG. 1 and described above. The first anatomical structure shown by the first ultrasound image 705 may be a LAA of a patient. Further, the first ultrasound image 705 may be obtained by the ultrasound imaging system 100 (e.g., the probe 106) while operating in the B-mode. In some embodiments, the first ultrasound image 705 may be one image of a group of images obtained sequentially by probe 106 while operating in the B-mode. The first ultrasound image 705 may be a static image or may be a series of images (e.g., video feed showing a cine loop). According to certain implementations, the first ultrasound image 705 displayed on the GUI may update in real-time as the ultrasound examination occurs and as more ultrasound data is collected by the probe 106.

As shown in FIGS. 7-8, the GUI 700 may include a display of imaging parameters 710. The imaging parameters 710 refer to imaging parameters of the ultrasound imaging system 100 applied during acquisition of the first ultrasound image 705. In some embodiments, the imaging parameters 710 may be default imaging parameters (e.g., unadjusted imaging parameters) associated with the B-mode. For example, and as shown, the imaging parameters may include a frame rate (FPS) (e.g., 48 frames per second), a frequency (e.g., 5.0 MHz), a power (e.g., −1 dB), a gain (e.g., 0 dB), a compression (e.g., 60 dB), a persistence (e.g., 0.7), and a depth (e.g., 12.0 cm) applied during the acquisition of the first ultrasound image 705.

Referring to FIG. 8, the GUI 700 is shown with the first ultrasound image 705, the imaging parameters 710, and options for next steps 800. That is, the options for next steps 800 refer to the operations (e.g., operations 410 generated by the entity recognition circuit 124) presented to the operator at step 535 of method 500, as described above. For example, continuing with the examples described herein and as shown in FIG. 8, the options for next steps 800 may include switching to a 0-degree LAA view, switching to a live 3D mode, switching to a color Doppler mode, and measuring a length of the LAA from the 90-degree view. As described above with reference to FIG. 5, the options for next steps 800 displayed on the GUI 700 may include an amount of detail based on operator settings/preferences. For example, a relatively new sonographer with less experience may prefer to receive a reason/explanation behind each of the options for next steps 800, whereas a more experienced sonographer may prefer to receive little to no additional details with the options for next steps 800 (e.g., as shown in FIG. 8).

Referring to FIGS. 9-10, a GUI 900 including a second ultrasound image 905 of the first anatomical structure obtained via the ultrasound imaging system is shown. In some embodiments, the GUI 700 may be displayed via the display device 132 of the ultrasound imaging system 100. The second ultrasound image 905 may be obtained by the ultrasound imaging system 100 shown by FIG. 1 and described above. The first anatomical structure shown by the second ultrasound image 905 is the LAA of the patient also shown in the first ultrasound image 705 of FIGS. 7-8. As shown in FIGS. 9-10, the second ultrasound image 905 may be obtained by the ultrasound imaging system 100 (e.g., the probe 106) while operating in the color Doppler mode. In some embodiments, the second ultrasound image 905 may be one image of a group of images obtained sequentially by probe 106 while operating in the color Doppler mode. The second ultrasound image 905 may be a static image or may be a series of images (e.g., video feed showing a cine loop). According to certain implementations, the second ultrasound image 905 displayed on the GUI may update in real-time as the ultrasound examination occurs and as more ultrasound data is collected by the probe 106.

As shown in FIG. 9, the GUI 900 includes the options for next steps 800 and a selection 915 from the options for next steps 800. The selection 915 refers to one of the options for next steps 800 that has been selected (e.g., tapped on, clicked on, or otherwise interacted with via the GUI 700) by the operator. That is, the selection 915 refers to the user selection of the operation executed during step 540 of method 500. For example, and as shown, the operator may select the option to switch to a color Doppler mode from the options for next steps 800. Therefore, the GUI 900 is configured to present the second ultrasound image 905 based on the choice to switch to the color Doppler mode.

Additionally, as shown in FIGS. 9 and 10, the GUI 900 includes updated imaging parameters 910 based on the selection 915. The updated imaging parameters 910 refer to imaging parameters of the ultrasound imaging system 100 applied during acquisition of the second ultrasound image 905. In some embodiments, the updated imaging parameters 910 may be default imaging parameters (e.g., unadjusted imaging parameters) associated with the color flow Doppler mode. For example, and as shown, the imaging parameters may include an FPS (e.g., 25 frames per second), a frequency (e.g., 3.6 MHz), a power (e.g., −1 dB), a gain (e.g., −3 dB), a left ventricle (LV) rejection (e.g., 14 cm/s), a scale (e.g., 6.00 kHz), and a sample volume (e.g., 0.5 mm) applied during the acquisition of the second ultrasound image 905.

While in the color Doppler mode, the ultrasound imaging system 100 may obtain grayscale information (e.g., monochromatic image data) as well as color information, such as color flow image data colored according to a color reference. The color reference may be used by the operator or other clinician (e.g., a cardiologist) in combination with a color flow mapping to determine the direction and/or speed of blood flowing within the anatomy being imaged (e.g., the LAA). The clarity of the color flow mapping (e.g., a resolution or amount of color gradation of the color flow mapping) may result from the values of the updated imaging parameters 910.

Referring to FIG. 9, the GUI 600, as described above with reference to FIG. 6, is also shown. The GUI 600 depicts a plurality of the selectable elements 605 (e.g., 605a, 605b, 605c, and 605d). In some embodiments, a number of the plurality of selectable elements 605 may correspond to a number of options for next steps requested in a natural language prompt received from the operator (e.g., natural language prompt 305, as described above with reference to FIG. 3). For example, if the operator requests three options for the next steps, and the AI circuit 122 outputs three options for the next steps, the GUI 600 may display three selectable elements 605 (e.g., 605a, 605b, and 605c). Furthermore, each of the plurality of selectable elements 605 may be configured to automatically perform, when engaged with by a user, one or more operations/functions of the ultrasound imaging system 100 associated therewith. For example, each of the three selectable elements 605 may be configured to automatically perform operations involved in a respective option from the three options for the next steps outputted by the AI circuit 122.

In some embodiments, the control circuit 126 may be configured to control (based on a control signal generated by the entity recognition circuit 124, as described above) the GUI 600 such that the GUI 600 displays a plurality of selectable elements 605 corresponding to operations of the ultrasound imaging system 100 (e.g., operations 410) involved in a plurality of recommended next steps (e.g., the textual description of next steps 320). Further, upon an indication that the operator has interacted/otherwise engaged with (e.g., chosen) one of the plurality of selectable elements 605, the control circuit 126 may be configured to execute operations associated with the chosen selectable element 605 (e.g., based on the control signal associated with the chosen selectable element 605).

As indicated by the dashed lines shown between the GUI 900 and the GUI 600 in FIG. 9, each of the plurality of selectable elements 605 corresponds to an option from the options for next steps 800. That is, selectable element 605a may be configured to automate the operation described by the first option from the options for next steps 800 (e.g., switching to a 0-degree LAA view), selectable element 605b may be configured to automate the operation described by the second option from the options for next steps 800 (e.g., switching to a live 3D mode), selectable element 605c may be configured to automate the operation described by the third option from the options for next steps 800 (e.g., switching to a color Doppler mode), and selectable element 605d may be configured to automate the operation described by the fourth option from the options for next steps 800 (e.g., measuring a length of the LAA from the 90-degree view). As shown in FIG. 9, selectable element 605c is depicted within a box to illustrate that the operator engaged with (e.g., pressed, clicked on, tapped, etc.) the selectable element 605c, which corresponds to the selection 915 (e.g., the third option from the options for next steps 800). Therefore, the ultrasound imaging system 100 may be prompted to switch to the color Doppler mode upon receiving the indication that the operator has engaged with selectable element 605c.

Referring to FIG. 10, GUI 900 is shown including the second ultrasound image 905, the updated imaging parameters 910, and updated options for next steps 1000. The updated options for next steps 1000 refer to new recommendations based on the updated status of the ultrasound imaging workflow (e.g., after switching from the B-mode to the color Doppler mode). As described above with reference to FIG. 5, the new recommendations may be generated during a second iteration of method 500 based on the updated status of the workflow. For example, the updated options for next steps 1000 may include switching to the 0-degree LAA view, switching to a PW mode, switching to the live 3D mode, and measuring the length of the LAA from the 90-degree view. Therefore, as shown in FIG. 10 and as described above with reference to FIG. 5, the updated options for next steps 1000 may include previously presented options for next steps (e.g., options 1, 2, and 4 from the options for next steps 800) that have not yet been selected by the operator. While the method 500 repeats until the operator has obtained a sufficient/complete ultrasound scan, the ultrasound imaging system 100 may be configured to generate additional graphical user interfaces configured to present intelligent guidance (e.g., options for next steps in the ultrasound imaging workflow) until the sufficient/complete scan is achieved.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. An ultrasound imaging system comprising:
an ultrasound probe comprising:
a transducer configured to transmit and receive an ultrasound signal;
a matching layer configured to have an acoustic impedance between a tissue to be imaged and a material of the transducer; and
a damping block configured to absorb ultrasound energy;
an image processing circuit configured to receive image data obtained using the ultrasound probe, and perform an image recognition process to identify an image characteristic based on the image data;
an entity recognition circuit configured to convert the identified image characteristic into an artificial intelligence (AI) model input;
an AI circuit comprising a retrieval model configured to receive contextual information, the contextual information comprising procedure-specific information and patient medical history;
the AI circuit configured to receive the AI model input, apply the AI model input and the contextual information to an AI model, and output an AI model output corresponding to a plurality of recommended next steps of a workflow process;
the entity recognition circuit configured to convert the AI model output into a control signal; and
a control circuit configured to:
receive the control signal;
receive an operator preference including a preference related to the plurality of recommended next steps;
update the plurality of recommended next steps based on the operator preference; and
control a display of the ultrasound imaging system, wherein controlling the display comprises causing the display to display the updated plurality of recommended next steps for an operator of the ultrasound imaging system to perform.

2. The ultrasound imaging system of claim 1, wherein the plurality of recommended next steps comprise at least one of a recommendation to rotate or move the ultrasound probe a certain way, a recommendation to activate a particular mode, or a recommendation to take a particular measurement.

3. The ultrasound imaging system of claim 1, where the control circuit is configured to change an operating characteristic of the ultrasound imaging system based on receiving a user input selecting one of the plurality of recommended next steps.

4. The ultrasound imaging system of claim 3, wherein changing the operating characteristic of the ultrasound imaging system comprises changing at least one of a mode, a view, an acquisition parameter, or a measurement setting.

5. The ultrasound imaging system of claim 1, wherein the control circuit is configured to display the plurality of recommended next steps in real-time with respect to the image processing circuit receiving the image data.

6. The ultrasound imaging system of claim 1, wherein the image characteristic comprises at least one of a mode, a view, an identification of an imaged structure, an identification of a pathology, an acquisition parameter, or a measurement setting.

7. The ultrasound imaging system of claim 1, wherein the AI model input comprises a natural language input.

8. The ultrasound imaging system of claim 1, wherein the AI model comprises a large language model.

9. (canceled)

10. The ultrasound imaging system of claim 8, wherein applying the AI model input to the AI model comprises:
querying the retrieval model configured to search a medical information database for data relating to the AI model input;
combining the data relating to the AI model input with the AI model input to create an augmented AI model input;
applying the augmented AI model input to the large language model; and
generating the AI model output.

11. The ultrasound imaging system of claim 10, wherein the medical information database comprises at least one of clinical guidelines, standard practices, medical literature, medical textbooks, published research, or previous case studies related to the AI model input.

12. The ultrasound imaging system of claim 1, wherein the AI model output comprises a set of natural language instructions, the set of natural language instructions including the plurality of recommended next steps.

13. An ultrasound imaging system comprising:
an ultrasound probe comprising:
a transducer configured to transmit and receive an ultrasound signal;
a matching layer configured to have an acoustic impedance between a tissue to be imaged and a material of the transducer; and
a damping block configured to absorb ultrasound energy; and
a processing circuit having a processor coupled to a memory device storing instructions thereon that, when executed, cause the processing circuit to perform operations comprising:
receiving image data obtained using the ultrasound probe;
performing an image recognition process to identify an image characteristic based on the image data;
converting the identified image characteristic into an artificial intelligence (AI) model input;
receiving, from a retrieval model, contextual information comprising procedure-specific information and patient medical history;
applying the AI model input and the contextual information to an AI model configured to generate an AI model output corresponding to a plurality of recommended next steps of a workflow process;
converting the AI model output into a control signal; and
controlling a display of the ultrasound imaging system based on the control signal, wherein controlling the display comprises causing the display to display the plurality of recommended next steps for an operator of the ultrasound imaging system to perform.

14. The ultrasound imaging system of claim 13, wherein the AI model comprises a large language model, and wherein applying the AI model input to the AI model comprises:
querying the retrieval model configured to search a medical information database for data relating to the AI model input;
combining the data relating to the AI model input with the AI model input to create an augmented AI model input;
applying the augmented AI model input to the large language model; and
generating the AI model output.

15. A method comprising:
receiving, by an image processing circuit, image data obtained using an ultrasound probe;
performing, by the image processing circuit, an image recognition process to identify an image characteristic based on the image data;
converting, by an entity recognition circuit, the identified image characteristic into an artificial intelligence (AI) model input;
receiving, by an AI circuit, the AI model input;
receiving, by a retrieval model of the AI circuit, contextual information comprising procedure-specific information and patient medical history;
applying, by the AI circuit, the AI model input and the contextual information to an AI model;
outputting, by the AI circuit, an AI model output corresponding to a plurality of recommended next steps of a workflow process;
converting, by the entity recognition circuit, the AI model output into a control signal;
receiving, by a control circuit, the control signal; and
controlling, by the control circuit, a display of an ultrasound imaging system based on the control signal, wherein controlling the display comprises causing the display to display the plurality of recommended next steps for an operator of the ultrasound imaging system to perform.

16. The method of claim 15, wherein the plurality of recommended next steps comprise at least one of a recommendation to rotate or move the ultrasound probe a certain way, a recommendation to activate a particular mode, or a recommendation to take a particular measurement.

17. The method of claim 15, further comprising changing, by the control circuit, an operating characteristic of the ultrasound imaging system based on receiving a user input selecting one of the plurality of recommended next steps, and wherein changing the operating characteristic of the ultrasound imaging system comprises changing at least one of a mode, a view, an acquisition parameter, or a measurement setting.

18. The method of claim 15, wherein the image characteristic comprises at least one of a mode, a view, an identification of an imaged structure, an identification of a pathology, an acquisition parameter, or a measurement setting.

19. The method of claim 15, wherein the AI model comprises a large language model and wherein the AI model input comprises a natural language input.

20. The method of claim 19, wherein applying, by the AI circuit, the AI model input to the AI model comprises:
querying, by the AI circuit, the retrieval model configured to search a medical information database for data relating to the AI model input;
combining, by the AI circuit, the data relating to the AI model input with the AI model input to create an augmented AI model input;
applying, by the AI circuit, the augmented AI model input to the large language model; and
generating, by the AI circuit, the AI model output.

21. The ultrasound imaging system of claim 1, wherein the AI model input comprises a current status and the AI circuit is further configured to:
receive a natural language prompt including a number of configurable options;
combine the natural language prompt including the number of configurable options with the AI model input to create an augmented AI model input;
apply the augmented AI model to the AI model to output an AI model output corresponding to the plurality of recommended next steps, the plurality of recommended next steps based on the received number of configurable options;
receive an updated status based on a performed next step from the number of configurable options of the plurality of recommended next steps; and
output an updated AI model output based on the updated status, such that the control circuit is configured to cause the display to display an updated plurality of recommended next steps based on the performed next step.
